(19) United States
(12) Patent Application Publication
(10) Pub. No.: US 2026/0144835 A1
(43) Pub. Date: May 28, 2026

(54) MULTIFUNCTIONAL NUTRACEUTICAL FOR ENHANCING FEMALE HEALTH

(71) Applicant: Robert R. Beltran, Rancho Santa Fe, CA (US)

(72) Inventor: Robert R. Beltran, Rancho Santa Fe, CA (US)

(21) Appl. No.: 18/961,919

(22) Filed: Nov. 27, 2024

(51) Int. Cl. A61K 36/185; A61K 31/197; A61K 31/198; A61K 31/315; A61K 31/355; A61K 31/375; A61K 31/4188; A61K 31/4406; A61K 31/51; A61K 31/519; A61K 31/522; A61K 31/525; A61K 31/593; A61K 31/675; A61K 35/00; A61K 35/741; A61K 36/07; A61K 36/15; A61K 36/16; A61K 36/258; A61K 36/296; A61K 36/31; A61K 36/48; A61K 36/53; A61K 36/67; A61K 36/71; A61K 36/73; A61K 36/81; A61K 36/82; A61K 36/899; A61K 36/9066

(52) U.S. Cl. CPC A61K 36/185; A61K 31/197; A61K 31/198; A61K 31/315; A61K 31/355; A61K 31/375; A61K 31/4188; A61K 31/4406; A61K 31/51; A61K 31/519; A61K 31/522; A61K 31/525; A61K 31/593; A61K 31/675; A61K 35/741; A61K 36/07; A61K 36/15; A61K 36/16; A61K 36/258; A61K 36/296; A61K 36/31; A61K 36/48; A61K 36/53; A61K 36/67; A61K 36/71; A61K 36/73; A61K 36/81; A61K 36/82; A61K 36/899; A61K 36/9066; A61K 2035/115

(57) ABSTRACT

A composition for improving female health, sexual performance, and benefiting the gut-brain axis is disclosed. The composition includes sufficient amounts of Black Cohosh Extract 20:1, Black Pepper Pepper Extract (95% piperine), Korean Red Ginseng 8:1, Ginkgo Biloba Leaf 8:1, Horny Goat Weed Extract 8:1, Tribulis Terrestris vine 8:1, Avena Sativa Extract Seed 8:1, Pine Bark Extract 8:1, Muira Pauma Extract 8:1, Lemon Balm P.E. 4:1 (Melissa officinalis folium), Red Raspberry Leaf Extract 4:1, Damiana Leaf Extract 4:1, Butea Superba Root 8:1, Ashwagandha Extract 4:1, L-Arginine HCL Powder, L-Citrulline, a Probiotic Blend, TKA 200, Fermented Green Tea Extract with L-Theanine and Caffeine, Folic Acid, Zinc Aspartate, Vitamin E (D-alpha Tocopheryl Acetate), Vitamin D3, Vitamin C (Ascorbic Acid), Vitamin B12 (Methyl cobalamin), Vitamin B6 (Pyridoxine HCL), Pantothenic Acid (D-Calcium Pantothenate), Vitamin B3 Crystalline Powder (Niacinamide), Vitamin B2 Powder (Riboflavin), Biotin (D-biotin), Vitamin B1 HCl (Thiamine HCL).

### TECHNICAL FIELD

Embodiments of the invention relate to compositions for a nutraceutical, and more specifically to a composition for improving overall female health including the advantages of improving the health and function of the gut-brain axis, enhancing sexual performance and enjoyment, improving digestive functions, improving mood, and enhancing cognitive performance.

### BACKGROUND

Recent research has shown that the human gut microbiome is an important factor in maintaining and even improving mental health, cognitive performance, increasing energy, and improving or maintaining overall wellbeing. While many supplements are marketing to provide these benefits, they often only contain few ingredients in ineffective or less-effective doses. In particular, it is known that improving the health and function of the gut-brain axis can result in various health benefits including enhanced sexual performance and enjoyment, improved digestive functions, improved mood, and improved cognitive performance.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

The embodiments described herein relate to a composition for improving health and function of the gut-brain axis, enhancing sexual performance and enjoyment, improving digestive functions, improving mood, enhancing cognitive performance, and providing mild to moderate pain and inflammation management.

The disclosed composition for improving female health, sexual performance, and benefiting the gut-brain axis includes sufficient amounts of Black Cohosh Extract 20:1, Black Pepper Root Extract 10% curcuminoids (Curcuma longa) (fermented), Korean Red Ginseng 8:1, Ginkgo Biloba Leaf 8:1, Horny Goat Weed Extract 8:1, Tribulis Terrestris vine 8:1, Avena Sativa Extract Seed 8:1, Pine Bark Extract 8:1, Muira Pauma Extract 8:1, Lemon Balm P.E. 4:1 (Melissa officinalis folium), Red Raspberry Leaf Extract 4:1, Damiana Leaf Extract 4:1, Butea Superba Root 8:1, Ashwagandha Extract 4:1, L-Arginine HCL Powder, L-Citrulline, a Probiotic Blend, Tongkat Ali 200, Reactivate CE, Fermented Green Tea Extract with L-Theanine and Caffeine, Folic Acid, Zinc Aspartate, Vitamin E (D-alpha Tocopheryl Acetate), Vitamin D3, Vitamin C (Ascorbic Acid), Vitamin B12 (Methyl cobalamin), Vitamin B6 (Pyridoxine HCL), Pantothenic Acid (D-Calcium Pantothenate), Vitamin B3 Crystalline Powder (Niacinamide), Vitamin B2 Powder (Riboflavin), Biotin (D-biotin), Vitamin B1 HCl (Thiamine HCL).

The composition provides a nutraceutical which is beneficial for various human cognitive and physical functions when administered at an effective dose. The composition may especially benefit female subjects. The effective dose may be administered via the oral route, such as by ingesting 2 capsules each containing equal, or about equal amounts of the composition.

Other objects and advantages of the various embodiments of the present invention will become obvious and intuitive to the reader and it is intended that these objects and advantages are within the scope of the present invention.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided here relate to a composition which may be administered to or by a human to enhance sexual performance and enjoyment, improve digestive functions, improve mood, improve cognitive performance, and provide mild to moderate pain and inflammation management. The composition may be beneficial to females which can experience the aforementioned benefits when the composition is administered at the effective dose. The composition includes various ingredients which provide these benefits at the specified amounts or ranges of amounts provided herein. The composition may be provided as a capsule, such that the active ingredients of the compound are encapsulated therein and may be administered by a sufficient route, such as by administering the capsule(s) orally.

In some embodiments, the composition includes sufficient amounts of the following: Black Cohosh Extract 20:1, Black Pepper Root Extract 10% curcuminoids (Curcuma longa) (fermented), Korean Red Ginseng 8:1, Ginkgo Biloba Leaf 8:1, Horny Goat Weed Extract 8:1, Tribulis Terrestris vine 8:1, Avena Sativa Extract Seed 8:1, Pine Bark Extract 8:1, Muira Pauma Extract 8:1, Lemon Balm P.E. 4:1 (Melissa officinalis folium), Red Raspberry Leaf Extract 4:1, Damiana Leaf Extract 4:1, Butea Superba Root 8:1, Ashwagandha Extract 4:1, L-Arginine HCL Powder, L-Citrulline, a Probiotic Blend, TKA 200, Fermented Green Tea Extract with L-Theanine and Caffeine, Folic Acid, Zinc Aspartate, Vitamin E (D-alpha Tocopheryl Acetate), Vitamin D3, Vitamin C (Ascorbic Acid), Vitamin B12 (Methyl cobalamin), Vitamin B6 (Pyridoxine HCL), Pantothenic Acid (D-Calcium Pantothenate), Vitamin B3 Crystalline Powder (Niacinamide), Vitamin B2 Powder (Riboflavin), Biotin (D-biotin), Vitamin B1 HCl (Thiamine HCL).

Fermented Green Tea Extract, L Theanine, and Caffeine is a bioavailable and “gut friendly” composition including all natural and fermented L-Theanine and green tea extract. The active ingredients are shown to increase reaction time, recall, and focus and can be used as a nootropic, psycho-biotic, pre-workout, and/or weight loss supplement. It may include green tea Extract which contains L-theanine and provide similar benefits to natural coffee. It is well known that these ingredients increase brain activity, energy, reaction time, recall, and focus.

Fermented Green Tea Extract, L Theanine, and Caffeine may provide a synergistic effect by combining caffeine and L-theanine which, when administered together, provides an unexpected benefit compared to administering each ingredient alone.

In some embodiments, Fermented Green Tea Extract, L Theanine, and Caffeine may have a suppressive effect on body weight and body fat accumulation.

In some embodiments, fermenting L-theanine provides the unexpected benefit of reducing “jitters” or other undesired side-effects of consuming L-theanine which has not been fermented.

The synergistic effects of caffeine and L-theanine have been shown to provide benefits which are experienced in a much more positive and effective way when compared to taking each ingredient separately from one another.

In some embodiments, administering fermented L-theanine provides a composition which is easily digestible and does not upset the gut, gut microbiome, or cause discomfort to the user.

Sufficient amounts of each of the active ingredients of the composition may be defined as amounts within the sufficient range of each ingredient as described herein.

In some embodiments, a sufficient amount of Black Cohash Extract 20:1 is between about 40-60 mg. In some embodiments, the sufficient amount of Black Cohash Extract is about 50 mg.

In some embodiments, a sufficient amount of Fermented Green Tea Extract, L Theanine, and Caffeine is between about 100-200 mg. In some embodiments, the sufficient amount of Fermented Green Tea Extract, L Theanine, and Caffeine is about 176 mg.

In some embodiments, a sufficient amount of Ashwagandha Extract is between about 40-60mg. The Ashwagandha Extract may be formulated as Ashwagandha Extract 4:1. In some embodiments, the sufficient amount of Ashwagandha Extract is 50 mg.

In some embodiments, a sufficient amount of Black Cohosh Extract is between about 25-100mg. The Black Cohosh Extract is formulated as Black Cohosh Extract 20:1. In some embodiments, the sufficient amount of Black Cohosh Extract is 50 mg.

In some embodiments, a sufficient amount of Black Pepper Extract (such as a formulation having 95% Piperine) is between about 3-7 mg. In some embodiments, the suitable amount of Black Pepper Extract may be about 5 mg.

In some embodiments, a sufficient amount of Turmeric Root Extract (such as a formulation having 10% curcuminoids (Curcuma longa) fermented) is between about 20-30 mg. In some embodiments, the sufficient amount of Turmeric Root Extract is 25 mg. The Turmeric Root Extract provides the benefits of pain and inflammation management.

In some embodiments, a sufficient amount of Maca Root is between about 16-24 mg and is formulated as Maca Root 8:1. In some embodiments, the sufficient amount of Maca Root is 20 mg.

In some embodiments, a sufficient amount of Lions Mane Extract (such as a formulation having 10% Polysaccharides) is between about 20-30 mg. In some embodiments, the sufficient amount of Lions Mane Extract is 25 mg.

In some embodiments, a sufficient amount of Korean Red Ginseng is between about 10 mg and 15 mg and is formulated as Korean Red Ginseng 8:1. In some embodiments, the sufficient amount of Korean Red Ginseng is about 12.5 mg.

In some embodiments, a sufficient amount of Ginkgo Biloba Leaf is between about 10-15 mg and is formulated as Ginkgo Biloba Leaf 8:1. In some embodiments the sufficient amount of Ginkgo Biloba Powder Extract is about 12.5 mg.

In some embodiments, a sufficient amount of Horny Goat Weed Extract is between about 5 mg and 7.5 mg and is formulated as Horny Goat Weed Extract 8:1. In some embodiments, a sufficient amount of Horny Goat Weed Extract is about 6.25 mg.

In some embodiments, a sufficient amount of Tribulis Terrestris vine is between about 10 mg and 15 mg and is formulated as Tribulis Terrestris vine 8:1. In some embodiments, a sufficient amount of Tribulis Terrestris vine is about 12.5 mg.

In some embodiments, a sufficient amount of Avena Sativa Extract Seed is between about 10-15 mg and is formulated as Avena Sativa Extract Seed 8:1. In some embodiments, the sufficient amount of Avena Sativa Extract Seed is 12.5 mg.

In some embodiments, a sufficient amount of Pine Bark Extract is between about 10-15 mg and is formulated as Pine Bark Extract 8:1. In some embodiments, the sufficient amount of Pine Bark Extract is about 12.5 mg.

In some embodiments, a sufficient amount of Muira Pauma Extract is between about 20-30 mg and is formulated as Muira Pauma Extract 8:1. In some embodiments, the sufficient amount of Muira Pauma Extract is about 25 mg.

In some embodiments, a sufficient amount of Lemon Balm is between about 40-60 mg and is formulated as Lemon Balm P.E. 4:1. In some embodiments, the sufficient amount of Lemon Balm is about 50 mg.

In some embodiments, a sufficient amount of Red Raspberry Leaf Extract is between about 40-60 mg and is formulated as Red Raspberry Leaf Extract 4:1. In some embodiments, the sufficient amount of Red Raspberry Leaf Extract is about 50 mg.

In some embodiments, a sufficient amount of Damiana Leaf Extract is between about 20-30 mg and is formulated as Damiana Lead Extract 4:1. In some embodiments, the sufficient amount of Damiana Leaf Extract is about 25 mg.

In some embodiments, a sufficient amount of Butea Superba Root is between about 16-24 mg and is formulated as Butea Superba Root 8:1. In some embodiments, the sufficient amount of Butea Superba Root is about 20 mg.

In some embodiments, a sufficient amount of L-Arginine HCL Powder is between about 240-360 mg and is more specifically provided at a dose of about 300 mg.

In some embodiments, a sufficient amount of L-Citruline is between about 160-240 mg and is more specifically provided at a dose of about 200 mg.

In some embodiments, a sufficient amount of a female Probiotic Blend (at 100 cfu) is between about 16-24 mg. In some embodiments, the sufficient amount of the female Cognitive Probiotic Blend is about 20 mg.

In some embodiments, the female probiotic blend (i.e., the cognitive matric) may include about 1263 mg in total of the following: Alpha Deum, Fermented Green Tea Extract, L Theanine, and Caffeine, Ashwagandha Extract 4:1, Acto-pro 10 (ginseng Extract 10%), gingko biloba Extract 24%, lion's mane extract 10%, Rhodiola rosea extract 3%, Rosavins 1%, turmeric extract 10%, cognitive probiotic (100 billion cfu) 2 billion cfu/serving, black pepper extract 95%, and Huperzine A 1%.

In some embodiments, a sufficient amount of Tongkat Ali 200 is between about 100-150 mg and may be provided at an amount of 125 mg.

In some embodiments, Reactivate CE (green teak extract and L-Theanine) are provided between 141-211 mg and may be provided at an amount of about 176 mg.

The cognitive blend (i.e., the cognitive matrix) may improve gastrointestinal health (i.e., gut health and gut microbiome health) and thus improve digestive functions. In addition, the gut-brain axis which is important for neurotransmitter replenishment, humoral function and immune response is optimized.

In some embodiments, a sufficient amount of TKA is between about 50-500 mg and is more specifically provided as a dose of about 125 mg.

In some embodiments, a sufficient amount of Folic Acid is about 0.2 mg.

In some embodiments, a sufficient amount of Zinc Apsartate is about 5.5 mg.

In some embodiments, a sufficient amount of Vitamin E (D-alpha Tocopheryl Acetate) is about 7.5 mg.

In some embodiments, a sufficient amount of Vitamin D3 is about 0.01 mg.

In some embodiments, a sufficient amount of Vitamin C (Ascorbic Acid) is between 36-54 mg. In some embodiments, the sufficient amount of Vitamin C is about 45 mg.

In some embodiments, a sufficient amount of Vitamin B6 (Pyridoxine HCL) is about 0.850 mg.

In some embodiments, a sufficient amount of Pantothenic Acid (D-Calcium Pantothenate) is between about 1.5-3.5 mg. In some embodiments, the sufficient amount of Pantothenic Acid is about 2.5 mg.

In some embodiments, a sufficient amount of Vitamin B3 Crystalline Powder (Niacinamide) is between about 5.6-8.4 mg. In some embodiments, the sufficient amount of Vitamin B3 Crystalline Powder is about 7 mg.

In some embodiments, a sufficient amount of Vitamin B2 Powder (Riboflavin) is about 0.650 mg.

In some embodiments, a sufficient amount of Vitamin B1 HCL (Thiamine HCL) is about 0.6 mg.

In some embodiments, a sufficient amount of Biotin is about 0.015 mg.

Each of the above ingredients of the composition may be encapsulated or otherwise prepared during a manufacturing process for administration to a human. The human will ingest the capsule via the oral route (or other sufficient administration route). In some embodiments, a single service size is two capsules. Each of the two capsules may include about equal amounts of the composition, such that ingesting the two capsules simultaneously or in reasonable succession will administer a single dose of the composition.

One skilled in the arts will readily understand that various capsule sizes and encapsulation techniques may be implemented. For example, a larger capsule size may only require a single capsule of the composition to administer the effective dose.

An effective dose of the composition is defined as an amount (such as the amount provided by the embodiments described herein) which results in one or more of the benefits of the composition. The benefits include improved cognitive performance, enhanced mental clarity, increased focus, and increased physical and mental energy.

The composition may include various inactive ingredients including, for example, Silicone Dioxide (Silica), Rice Flour, and Magnesium Stearate.

In some embodiments, the composition is formulated as a powder. Each ingredient of the composition is mixed, and otherwise processes, to create a mixed powder prior to the encapsulation process. Capsules may include vegetable bases capsules, or other encapsulation means known in the arts. While the example of a serving size of 2 capsules is used herein, it is to be understood that various capsule sizes may be used, which may result in variable amount of capsules within a particular bottle and the variability in the number of capsules needed to reach the effective dose.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein. It will be understood that it would be unduly repetitious and obfuscating to literally describe every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of explaining and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof may or may not have been described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A composition for enhancing female health, increasing female libido, increasing occurrence, intensity and duration of female orgasm, the composition comprising:
a sufficient amount of Black Cohash Extract;
a sufficient amount of Fermented Green Tea Extract, L Theanine, and Caffeine;
a sufficient amount of Black Cohosh Extract;
a sufficient amount of Black Pepper Extract;
a sufficient amount of Tumeric Root Extract;
a sufficient amount of Maca Root;
a sufficient amount of Lions Mane Extract;
a sufficient amount of Korean Red Ginseng;
a sufficient amount of Ginkgo Biloba Powder Extract;
a sufficient amount of Horny Goat Weed Extract;
a sufficient amount of Tribulis Terrestris vine:
a sufficient amount of Avena Sativa Extract Seed
a sufficient amount of a female Probiotic Blend;
a sufficient amount of Pine Bark extract;
a sufficient amount of Muira Pauma Extract;
a sufficient amount of Lemon Balm P.E.;
a sufficient amount of Red Raspberry Leaf Extract;
a sufficient amount of Damiana Lead Extract;
a sufficient amount of Butea Superba Root;
a sufficient amount of Ashwagandha Extract;
a sufficient amount of Folic Acid;
a sufficient amount of Zinc Aspartate;
a sufficient amount of L-Citruline;
a sufficient amount of Tongkat Ali 200;
a sufficient amount of Reactivate CE;
a sufficient amount of L-Arginine HCL powder;
a sufficient amount of Vitamin E;
a sufficient amount of Vitamin D3;
a sufficient amount of Vitamin C;
a sufficient amount of Vitamin B12;
a sufficient amount of Vitamin B6:
a sufficient amount of Pantothenic Acid;
a sufficient amount of Vitamin B3 Crystalline Powder;
a sufficient amount of Vitamin B2 Powder;
a sufficient amount of Vitamin B1 HCL; and
a sufficient amount of Biotin.

2. The composition of claim 1, wherein the composition is manufactured as a capsule.

3. The composition of claim 2, wherein an effective dose of the composition is two capsules.

4. The composition of claim 3, wherein the effective dose includes each of the following: about 50 mg of Black Cohash Extract, about 176 mg of Fermented Green Tea Extract, L Theanine, and Caffeine; about 50 mg of Ashwagandha Extract; about 5 mg of Black Pepper Extract; about 50 mg of Black Cohosh Extract; about 25 mg of Tumeric Root Extract; about 20 mg of Maca Root Extract; about 25 mg of Lions Mane Extract; about 12.5 mg of Ginkgo Biloba Leaf; about 12.5 mg of Korean Red Ginseng; about 6.25 mg of Horny Goat Weed Extract; about 20 mg of the female Probiotic Blend; about 12.5 mg of Tribulis Terrestris vine; about 12.5 mg of Avena Sativa Extract Seed; about 12.5 mg of Pine Bark Extract; about 25 mg of Muira Pauma Extract; about 50 mg of Lemon Balm P.E.; about 50 mg of Red Raspberry Leaf Extract; about 25 mg of Damiana Leaf Extract; about 20 mg of Butea Superba Root; about 300 mg of L-Arginine HCL Powder; about 200 mg of L-Citruline; 0.2 mg of Folic Acid; about 0.01 mg of Vitamin D3; about 45 mg of Vitamin C; about 0.001 mg of Vitamin B12; about 0.850 mg of Vitamin B6; about 2.5 mg of Pantothenic Acid; about 7 mg of Vitamin B3 Crystalline Powder; about 0.65 mg of Vitamin B2 Powder; about 0.6 mg of Vitamin B1 HCL; and about 0.015 mg of Biotin.

5. The composition of claim 4, wherein composition is formulated as a powder, and wherein the powder is mixed and encapsulated using a capsule to provide the effective dose upon ingestion of at least one capsule.

6. The composition of claim 5, wherein the sufficient amount of fermented Green Tea Extract, L Theanine, and Caffeine provides a synergistic benefit.

7. The composition of claim 6, wherein the sufficient amount of fermented Green Tea Extract, L Theanine, and Caffeine provide a suppressive effect on body weight and body fat.

8. The composition of claim 7, wherein the sufficient amount of fermented Green Tea Extract, L Theanine, and Caffeine promote brain activity, energy, and increase sexual pleasure.

9. The composition of claim 8, wherein the composition is utilized as sexual enhancement supplement.

10. A composition for improving female health and sexual pleasure, increasing female
libido, increasing occurrence, intensity and duration of female orgasm, the composition comprising:
a sufficient amount of Fermented Green Tea Extract, L Theanine, and Caffeine, wherein the sufficient amount of the Fermented Green Tea Extract, the L Theanine and the Caffeine is between 100-200 mg.
a sufficient amount of Ashwagandha Extract, wherein the sufficient amount of Ashwagandha Extract is between about 40-60 mg;
a sufficient amount of Black Pepper Extract wherein the sufficient amount of Black Pepper Extract is between about 4-6 mg;
a sufficient amount of Black Cohosh Extract, wherein the sufficient amount of Black Cohosh Extract is between about 25-100 mg;
a sufficient amount of Turmeric Root Extract wherein the sufficient amount of Turmeric Root Extract is between about 20-30 mg;
a sufficient amount of Maca Root, wherein the sufficient amount of Maca Root is between about 16-24 mg;
a sufficient amount of Lions Mane Extract, wherein the sufficient amount of Lions Mane Extract is between about 20-30 mg;
a sufficient amount of Ginkgo Biloba Powder Leaf, wherein the sufficient amount of Ginkgo Biloba Leaf Extract is between about 10-15 mg;
a sufficient amount of Horny Goat Weed Extract, wherein the sufficient amount of Horny Goat Weed Extract is about 5-7.5 mg;
a sufficient amount of Tribulis Terrestris vine, wherein the sufficient amount of Rhodiola Extract is between about 10-15 mg:
a sufficient amount of Pine Bark Extract, wherein the sufficient amount of Pine Bark Extract is between about 10-15 mg;
a sufficient amount of Muira Pauma Extract, wherein the sufficient amount of Muira Pauma Extract is between about 10-100 mg;
a sufficient amount of Lemon Balm, wherein the sufficient amount of Lemon Balm is between about 40-60 mg;
a sufficient amount of Red Raspberry Leaf Extract, wherein the sufficient amount of Red Raspberry Leaf Extract is between about 40-60 mg;
a sufficient amount of Damiana Leaf Extract, wherein the sufficient amount of Damiana Leaf Extract is between about 20-30 mg;
a sufficient amount of Butea Superba Root, wherein the sufficient amount of Butea Superba Root is between about 16-24 mg;
a sufficient amount of L-Arginine HCL Powder, wherein the sufficient amount of L-Arginine HCL Powder is between about 240-360 mg;
a sufficient amount of L-Citruline, wherein the sufficient amount of L-Citruline is between about 160-240 mg;
a sufficient amount of Tongkat Ali 200, wherein the sufficient amount of Tongkat Ali 200 is between about 100-150 mg;
a sufficient amount of Reactivate CE, wherein the sufficient amount of Reactive CE is between about 141-211 mg;
a sufficient amount of TKA, wherein the sufficient amount of TKA is between about 50-500 mg;
a sufficient amount of a female Cognitive Probiotic Blend, wherein the sufficient amount of the Cognitive Probiotic Blend is between about 16-24 mg;
a sufficient amount of Folic Acid, wherein the sufficient amount of Folic Acid is about 0.2 mg;
a sufficient amount of Vitamin D3, wherein the sufficient amount of Vitamin D3 is about 0.01 mg;
a sufficient amount of Vitamin C Powder, sufficient amount of Vitamin C (Ascorbic Acid) is between 36-54 mg;
a sufficient amount of Vitamin B12, wherein the sufficient amount of Vitamin B12 is about 0.001 mg;
a sufficient amount of Vitamin B6, wherein the sufficient amount of Vitamin B6 is about 0.850 mg;
a sufficient amount of Pantothenic Acid, wherein the sufficient amount of Pantothenic Acid is between about 2-3 mg:
a sufficient amount of Vitamin B3 Crystalline Powder, wherein the sufficient amount of Vitamin B3 Crystalline Powder is between about 5.6-8.4 mg;
a sufficient amount of Vitamin B2 Powder, wherein the sufficient amount of Vitamin B2 Powder is between about 0.650 mg. a sufficient amount of Vitamin B1 HCL, wherein the sufficient amount of Vitamin B1 HCL is about 0.6 mg; and
a sufficient amount of Biotin, wherein the sufficient amount of Biotin is about 0.015 mg.

11. The composition of claim 10, wherein the composition is manufactured as a capsule.

12. The composition of claim 11, wherein an effective dose of the composition is two capsules.

13. The composition of claim 10, wherein the effective dose includes each of the following: about 176 mg of Fermented Green Tea Extract, L Theanine, and Caffeine; about 50 mg of Ashwagandha Extract; about 5 mg of Black Pepper Extract; about 50 mg of Black Cohosh Extract; about 25 mg of Tumeric Root Extract; about 20 mg of Maca Root Extract; about 25 mg of Lions Mane Extract; about 12.5 mg of Ginkgo Biloba Leaf; about 12.5 mg of Korean Red Ginseng; about 6.25 mg of Horny Goat Weed Extract; about 20 mg of the female Probiotic Blend; about 12.5 mg of Tribulis Terrestris vine; about 12.5 mg of Avena Sativa Extract Seed; about 12.5 mg of Pine Bark Extract; about 25 mg of Muira Pauma Extract; about 50 mg of Lemon Balm P.E.; about 50 mg of Red Raspberry Leaf Extract; about 25 mg of Damiana Leaf Extract; about 20 mg of Butea Superba Root; about 300 mg of L-Arginine HCL Powder; about 200 mg of L-Citruline; 0.2 mg of Folic Acid; about 0.01 mg of Vitamin D3; about 45 mg of Vitamin C; about 0.0001 mg of Vitamin B12; about 0.850 mg of Vitamin B6; about 2.5 mg of Pantothenic Acid; about 7 mg of Vitamin B3 Crystalline Powder; about 0.65 mg of Vitamin B2 Powder; about 0.6 mg of Vitamin B1 HCL; and about 0.015 mg of Biotin.

14. The composition of claim 13, wherein composition is formulated as a powder, and wherein the powder is mixed and encapsulated using a capsule to provide the effective dose upon ingestion of at least one capsule.

15. The composition of claim 14, wherein the sufficient amount of fermented Green Tea Extract, L Theanine, and Caffeine provides a synergistic benefit.

16. The composition of claim 15, wherein the sufficient amount of fermented Green Tea Extract, L Theanine, and Caffeine provide a suppressive effect to increase female health.

17. The composition of claim 16, wherein the sufficient amount of fermented Green Tea Extract, L Theanine, and Caffeine promote increased sexual pleasure.

18. The composition of claim 17, wherein the composition is utilized as a means of enhancing the functions of the gut-brain axis.

19. The composition of claim 18, wherein the composition is utilized as a psycho-biotic supplement.

20. The composition of claim 19, wherein the composition is utilized as a women's health supplement.
